(19) United States
(12) Patent Application Publication
(10) Pub. No.: US 2026/0216290 A1
(43) Pub. Date: Jul. 30, 2026

(54) USE OF AMPHIREGULIN (AREG) IN METHODS OF TREATING VASCULAR HYPERPERMEABILITY

(71) Applicants: INSTITUT NATIONAL DE LA SANTÉ ET DE LA RECHERCHE MÉDICALE, Paris (FR); ASSISTANCE PUBLIQUE-HÔPITAUX DE PARIS (APHP), Paris (FR); CENTRE NATIONAL DE LA RECHERCHE SCIENTIFIQUE, Paris (FR); UNIVERSITÉ PARIS CITÉ, Paris (FR); COLLÈGE DE FRANCE, Paris (FR)

(72) Inventors: Stéphane GERMAIN, Paris (FR); Nicolas BRECHOT, Paris (FR)

(21) Appl. No.: 19/146,679

(22) PCT Filed: Jan. 26, 2024
(86) PCT No.: PCT/EP2024/051838

§ 371 Date: Jul. 9, 2025

(30) Foreign Application Priority Data
Jan. 27, 2023 (EP) 23305104.4

(51) Int. Cl. A61K 38/19; A61P 9/14

(52) U.S. Cl. CPC A61K 38/19; A61P 9/14

(57) ABSTRACT

The disruption of endothelial barrier integrity leading to increased vascular permeability contributes to many pathological processes. In a first cohort of 11 patients having cardiac arrest with vascular leakage, the inventors identified that AREG is 56-fold more expressed than control. During a second prospective clinical study, they confirmed a strong association between the plasma level of AREG and the fluid balance in 77 independent patients hospitalized for a cardiogenic shock. The inventors finally showed a crucial role for this protein in Areg−/− mice, with i) no KO-mice achieving a return of spontaneous circulation (ROSC) vs. 40% in WT mice (n=6 per group, p<0.05) and ii) a beneficial effect of 10 μg recombinant AREG i.v. injection at the time of resuscitation, with 80% injected mice that achieve a ROSC vs 40% in controls (n=6, p<0.05). Finally, using markers of vascular extravasation, they demonstrated that AREG administration was decreasing the level of vascular leakage. The results thus indicate that administering AREG in a patient suffering from vascular permeability would be beneficial. Thus, the present invention relates to the use of amphiregulin (AREG) in methods of treating vascular permeability.

### FIELD OF THE INVENTION

The present invention is in the field of medicine, in particular vascular diseases.

### BACKGROUND OF THE INVENTION

The endothelial barrier plays an essential role in the blood circulation and the exchanges between blood and tissues. Its permeability allowing these exchanges is mainly regulated by adhesion molecules such as tight junctions. Studies demonstrated that a dysregulation of these cellular junctions involved an increase of the vascular permeability associated or not with a structure alteration of endothelial barrier cells (Wautier J. L et al., 2022, Int J Mol Sci).

The disruption of endothelial barrier integrity leading to increased vascular permeability contributes to many pathological processes, some of which unfold quickly, within minutes or hours. For instance, conditions wherein acute vascular permeability are detrimental are circulatory shocks, that are responsible for ⅓ of intensive care unit (ICU) admissions (20,000/year in France) with 40% mortality and thus represent a major public health issue. Vascular hyperpermeability (capillary leakage) is indeed a major feature of circulatory failure. Inflammation triggered by tissue lesions (SIRS: systemic inflammatory response syndrome) also induces massive vascular leakage, which affects both macro and micro-circulation. Fluid balance (difference between fluid input and output) independently correlates with mortality during septic and cardiogenic shock (Besnier, E., et al. (2020). Shock 53, 426-433. 2 Micek, S. T., et al. (2013). Crit Care 17, R246. 3; Liu, L., et al. (2018). Basic Res Cardiol 113, 12.) and controlling capillary leakage would be highly beneficial. Many studies shown that diseases such as chronic inflammation, cancer, septic shock, diabetes or acute hemorrhagic shock induces an increase of the vascular permeability (Nagy J. A et al., 2008, Angiogenis; Pickkers P et al., 2005, Shock; Yuan S. Y et al., 2007, Microcirculation & Xia Z. L et al., 1995, Respiration). Then, it is known in the prior art that the vascular permeability is essential for the health of normal tissues and is also an important characteristic of many disease states in which it is greatly increased (Nagy J. A et al., 2008, Angiogenis). A vascular hyperpermeability induces biological dysfunction such as peripheral edema, tissue damage or fluid accumulation in the lungs (Agostoni A et al., 1992, Int J Clin Lab; Paul R et al., 2001, Nat Med; Weis S et al., 2004, J Clin Invest & Groeneveld A. B. J et al., 2002, Vascu Pharmacol).

The detrimental effects associated with vascular permeability thus necessitate the development of therapies and therapeutic agents that can effectively and timely prevent, reduce, or counteract the vascular permeability and protect tissues from ischemia/reperfusion injuries.

Amphiregulin (AREG) is a type-II cytokine and member of the epidermal growth factor family. The name AREG was derived because of its bifunctional role in stimulating the growth of keratinocytes, normal fibroblasts, as well as tumor cells, and inhibiting the proliferation of several invasive cancer cell lines in vitro (Shoyab, Mohammed, et al. “Amphiregulin: a bifunctional growth-modulating glycoprotein produced by the phorbol 12-myristate 13-acetate-treated human breast adenocarcinoma cell line MCF-7.” Proceedings of the National Academy of Sciences 85.17 (1988): 6528-6532.). It is synthesized as a transmembrane propeptide, released as mature AREG after proteolytic cleavage and can act in juxtacrine, autocrine, or paracrine manners. After engagement with EGF receptors (EGFR), AREG activates essential cascades of intracellular signaling governing cellular metabolism, inflammation, and cell cycle. The elevated expression of AREG is associated with different inflammatory and pathological conditions. For instance, AREG has been identified as a key regulatory factor secreted by both innate and adaptive immune cells, which not only promote the host resistance to pathogenic helminths but also assist in tissue repair and wound healing under different inflammatory conditions. Recombinant AREG has been shown to enhance the process of tissue repair in several models of infection-mediated injuries (Monticelli et al. “Innate lymphoid cells promote lung-tissue homeostasis after infection with influenza virus”, Nature Immunology 12(11), 2011); Burzyn et al., “A Special Population of Regulatory T Cells Potentiates Muscle Repair”, Cell 155, 1282-1295, 2013; Jamieson et al. Role of tissue 25 protection in lethal respiratory viral-bacterial coinfection Science 340(6137): 1230-12342013 2013; Jin, Richard M., Jordan Warunek, and Elizabeth A. Wohlfert. “Therapeutic administration of IL-10 and amphiregulin alleviates chronic skeletal muscle inflammation and damage induced by infection.” Immunohorizons 2.5 (2018): 142-154; Minutti et al. “A Macrophage-Pericyte Axis Directs Tissue Restoration via Amphiregulin-Induced Transforming Growth Factor Beta Activation” Immunity 50, 645-654, 2019). These studies highlight the role of AREG in promoting tissue repair during the recovery period by inducing cell proliferation and differentiation. AREG has been identified as a biomarker as well as a therapeutic target whereby preventing AREG activity is explored as a therapeutic approach in the context of various cancers and chronic inflammatory and fibrotic conditions (Singh, Siddharth S., et al. “Amphiregulin in cellular physiology, health, and disease: Potential use as a biomarker and therapeutic target.” Journal of Cellular Physiology 237.2 (2022): 1143-1156.). Likewise, in cardiovascular diseases, AREG was described as promoting survival, differentiation and proliferation of cardiac cells (WO2006081190). Pretreatment with AREG was also described as providing cardioprotection from ischemia and reperfusion injury in mice (Koeppen, Michael, et al. “Hypoxia-inducible factor 2-alpha-dependent induction of amphiregulin dampens myocardial ischemia-reperfusion injury.” Nature communications 9.1 (2018): 1-13).

However the specific interest of inducing or administering AREG as an early measure to promptly counteract damages caused by vascular permeability has never been envisaged and explored.

### SUMMARY OF THE INVENTION

The present invention is defined by the claims. In particular, the present invention relates to the use of amphiregulin (AREG) in methods of treating vascular permeability.

### DETAILED DESCRIPTION OF THE INVENTION

### Main Definitions

As used herein, the terms “polypeptide”, “peptide”, and “protein” are used interchangeably herein to refer to polymers of amino acids of any length. The terms also encompass an amino acid polymer that has been modified; for example, disulfide bond formation, glycosylation, lipidation, phosphorylation, or conjugation with a labeling component. Polypeptides when discussed in the context of gene therapy refer to the respective intact polypeptide, or any fragment or genetically engineered derivative thereof, which retains the desired biochemical function of the intact protein.

As used herein, the term “polynucleotide” or “nucleic acid” refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits. The promoter of the present invention can be prepared by any method known to one skilled in the art, including chemical synthesis, recombination, and mutagenesis. In particular, the promoter of the present invention is a DNA molecule, typically synthesized by recombinant methods well known to those skilled in the art.

As used herein, the expression “derived from” refers to a process whereby a first component (e.g., a first polypeptide or polynucleotide), or information from that first component, is used to isolate, derive or make a different second component (e.g., a second polypeptide or polynucleotide that is different from the first one).

As used herein, the term “encoding” refers to the inherent property of specific sequences of nucleotides in a polynucleotide, such as, for example, a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleic acid sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleic acid sequence encoding an amino acid sequence” includes all nucleic acid sequences that are degenerate versions of each other and that encode the same amino acid sequence.

A used herein, the term “Amphiregulin” or “AREG” has its general meaning in the art and refers to the protein synthesized as a transmembrane glycoprotein by the AREG gene (Shoyab, Mohammed, et al. “Structure and function of human amphiregulin: a member of the epidermal growth factor family.” Science 243.4894 (1989): 1074-1076.). The term is also know as AR; AREG; AREGB; Colorectum cell-derived growth factor; CRDGF; MGC13647; schwannoma-derived growth factor; or SDGF. AREG is transcribed as a 1.4-kb mRNA containing six exons and code for a membrane-anchored precursor protein of 252 amino-acids referred as pro-AREG. This precursor AREG protein contains many glycosylation motifs and cleavage sites leading to different mature AREG proteins and influence AREG's biological activity in different cell types. Sequence analysis has shown the presence of N-terminal domain with six spatially conserved cysteines and many other semiconserved amino acid residues, which form disulfide bridges to give rise to a three-looped structure, called EGF domain, implicated in binding to the EGFR. The subsequent proteolytic cleavage of the precursor mediated by the metalloproteinase enzyme TACE or ADAM-17 causes the release of the mature soluble AREG containing EGF motif, which subsequently induces autocrine or paracrine activation of EGFR leading to a cascade of signaling events required for several cellular processes including cell cycle, proliferation, and metabolism. An exemplary amino acid sequence for AREG is show as SEQ ID NO:1. The amino sequence of the EGF domain ranges from the amino acid residue at position 142 to the amino acid residue at position 182 in SEQ ID NO:1. As used herein, the term “AREG polypeptide” refers to a polypeptide that derives from AREG and that comprises the EGF domain of AREG or a functional variant thereof.

>sp|P15514|AREG_HUMAN Amphiregulin OS = Homo
sapiens OX = 9606 GN = AREG PE = 1 SV = 2.
The EGF domain is indicated in bold and is
underlined. The “recombinant” human
amphiregulin is indicated as underlined.
SEQ ID NO: 1
MRAPLLPPAPVVLSLLILGSGHYAAGLDLNDTYSGKREPFSGDHSA
DGFEVTSRSEMSSGSEISPVSEMPSSSEPSSGADYDYSEEYDNEPQ
IPGYIVDDSVRVEQVVKPPQNKTESENTSDKPKRKKKGGKNGKNRR
NRKKKNPCNAEFQNFCIHGECKYIEHLEAVTCKCQQEYFGERCGEK
SMKTHSMIDSSLSKIALAAIAAFMSAVILTAVAVITVQLRRQYVRK
YEGEAEERKKLRQENGNVHAIA

As used herein, the term “variant” refers to a amino acid sequence sequence differing from the original amino acid sequence, but retaining essential properties thereof. Generally, variants are overall closely similar, and, in many regions, identical to the original polypeptide. The sequence of the variant may differ by amino acid substitutions, deletions or insertions of one or more amino acid residues in the sequence, which do not impair the activity of the polypeptide. The variant may have the same length of the original sequence, or may be shorter or longer.

As used herein, the term “functional variant of the EGF domain of AREG” refers to a variant of the amino acid sequence that ranges from the amino acid residue at position 142 to the amino acid residue at position 182 in SEQ ID NO:1 and that is capable of binding to EGFR which subsequently induces autocrine or paracrine activation of EGFR leading to a cascade of signaling events required for several cellular processes including cell cycle, proliferation, and metabolism (Berasain, Carmen, and Matias A. Avila. “Amphiregulin.” Seminars in cell & developmental biology. Vol. 28. Academic Press, 2014.). Assays for assessing said functionality are well know in the art and typically include those described in Macdonald-Obermann, Jennifer L., and Linda J. Pike. “Different epidermal growth factor (EGF) receptor ligands show distinct kinetics and biased or partial agonism for homodimer and heterodimer formation.” Journal of Biological Chemistry 289.38 (2014): 26178-26188.

As used herein, the “percent identity” between the two sequences is a function of the number of identical positions shared by the sequences (i.e., % identity=number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described below. The percent identity between two amino acid sequences can be determined using the Needleman and Wunsch algorithm (Needleman, Saul B. & Wunsch, Christian D. (1970). “A general method applicable to the search for similarities in the amino acid sequence of two proteins”. Journal of Molecular Biology. 48 (3): 443-53.). The percent identity between two nucleotide or amino acid sequences may also be determined using for example algorithms such as EMBOSS Needle (pair wise alignment; available at www.ebi.ac.uk). For example, EMBOSS Needle may be used with a BLOSUM62 matrix, a “gap open penalty” of 10, a “gap extend penalty” of 0.5, a false “end gap penalty”, an “end gap open penalty” of 10 and an “end gap extend penalty” of 0.5. In general, the “percent identity” is a function of the number of matching positions divided by the number of positions compared and multiplied by 100. For instance, if 6 out of 10 sequence positions are identical between the two compared sequences after alignment, then the identity is 60%. The % identity is typically determined over the whole length of the query sequence on which the analysis is performed. Two molecules having the same primary amino acid sequence or polynucleic acid sequence are identical irrespective of any chemical and/or biological modification. According to the invention a first amino acid sequence having at least 80% of identity with a second amino acid sequence means that the first sequence has 80; 81; 82; 83; 84; 85; 86; 87; 88; 89; 90; 91; 92; 93; 94; 95; 96; 97; 98; 99 or 100% of identity with the second amino acid sequence.

As used herein, the term “patient” or “patient in need thereof”, is intended for a human or non-human mammal. Typically, the patient is affected or likely to be affected with vascular permeability.

As used herein, the term “vascular permeability” has its general meaning in the art and indicates the capability of passing through the blood vessels, which is functioning to regulate the blood-spill into the extracellular matrix of the vascular endothelial cells. More particularly, the term refers to the escape of blood plasma through capillary walls, from the blood circulatory system to surrounding tissues, muscle compartments, organs or body cavities. The terms “vascular permeability” and “vascular leakage” are used interchangeably herein. The term encompasses “excessive vascular permeability” and “hyperpermeability”. In particular, may be characterized by an alteration of the endothelial cellular adhesion molecule regulation and/or an endothelial cell structure alteration.

As used herein, the term “vascular permeability-associated disease or condition” refers to any disease or condition that results from, results in, is characterised by, or otherwise associated with vascular permeability (typically excessive vascular permeability or hyperpermeability). Thus, the association between the disease or condition and vascular permeability may be direct or indirect and may be temporally and/or spatially separated. In the context of the present specification the terms vascular permeability or excessive vascular permeability and vascular leak may be used interchangeably. In particular, the term “vascular permeability-associated disease or condition “indicates the disease caused by the failure of normal vascular permeability regulation.

As used herein, the term “capillary leak syndrome” or “vascular leak syndrome” has its general meaning in the art and refers to a syndrome that is characterized by the escape of blood plasma through capillary walls, from the blood circulatory system to surrounding tissues, muscle compartments, organs or body cavities. It is a phenomenon most commonly witnessed in sepsis and other forms of circulatory failure, and less frequently in autoimmune diseases, differentiation syndrome, engraftment syndrome, hemophagocytic lymphohistiocytosis, the ovarian hyperstimulation syndrome, viral hemorrhagic fevers, and snakebite and ricin poisoning. Pharmaceuticals, including the chemotherapy medications gemcitabine and denileukin diftitox, as well as certain interleukins and monoclonal antibodies, can also cause capillary leaks. These conditions and factors are sources of secondary capillary leak syndrome.

As used herein, the term “systemic capillary leak syndrome” is also called “Clarkson's disease”, or “primary capillary leak syndrome”, is a rare, grave and episodic medical condition observed largely in otherwise healthy individuals mostly in middle age. It is characterized by self-reversing episodes during which the endothelial cells which line the capillaries, usually of the extremities, separate for one to three days, causing a leakage of plasma mainly into the muscle compartments of the arms and legs. The abdomen, the central nervous system, and the organs (including the lungs) are typically spared, but the extravasation in the extremities is sufficiently massive to cause circulatory shock and compartment syndromes, with a dangerous hypotension (low blood pressure), hemoconcentration (thickening of the blood) and hypoalbuminemia (drop in albumin, a major protein) in the absence of other causes for such abnormalities.

As used herein, the term “vascular endothelial cell barrier” refers to the layer of cells that line the interior surface of blood vessels and act as a selective barrier between the vessel lumen and surrounding tissue, by controlling the transit of fluids, materials and cells such as myeloid cells and white blood cells into and out of the bloodstream. Excessive or prolonged increases in permeability of vascular endothelial cell barrier leads to tissue oedema/swelling. Accordingly the expression “preservation of vascular endothelial cell barrier integrity” means the maintenance of the vascular endothelial cell barrier by avoiding or limiting permeability of said barrier.

As used herein, the term “ischemic condition” has its general meaning in the art and refers to any condition that result from a restriction in blood supply in at least one organ or tissue. Ischemic condition typically results from the obstruction of a blood vessel. For example ischemic conditions include but are not limited to renal ischemia, retinal ischemia, brain ischemia and myocardial ischemia. More particularly, the term includes but it is not limited to coronary artery bypass graft surgery, global cerebral ischemia due to cardiac arrest, focal cerebral infarction, cerebral hemorrhage, hemorrhage infarction, hypertensive hemorrhage, hemorrhage due to rupture of intracranial vascular abnormalities, subarachnoid hemorrhage due to rupture of intracranial arterial aneurysms, hypertensive encephalopathy, carotid stenosis or occlusion leading to cerebral ischemia, cardiogenic thromboembolism, stroke, spinal stroke and spinal cord injury, diseases of cerebral blood vessels: e.g., atherosclerosis, vasculitis, macular degeneration, myocardial infarction, cardiac ischemia and superaventicular tachyarrhytmia. Alternatively, ischemia of all those organs can be caused by circulatory failure, without vascular obstruction. Those conditions include cardiogenic shock, sepsis and septic shock, hemorragic and anaphylactic shocks, as well as post-resuscitation (or post cardiac arrest syndrome) (Mehta S., Granton J., Gordon A. C., Cook D. J., Lapinsky S., Newton G., et al. “Cardiac ischemia in patients with septic shock randomized to vasopressin or norepinephrine”. Crit Care. 2013 Jun. 20; 17(3):R117; Geri G., Grimaldi G., Seguin T., Lamhaut L., Marin N., Chiche J. D., et al. “Hemodynamic efficiency of hemodialysis treatment with high cut-off membrane during the early period of post-resuscitation shock: The Hyperdia trial”. Resuscitation. 2019 July; 140:170-177; Haertel F., Reisberg D., Peters M., Nuding S., Schroeder J., Werdan K., and Ebelt H. “Prognostoc value of tissue oxygen saturation using a vascular occlusion test inpatients in the early phase of multiple organ dysfunction syndrome”. Shock. 2019 June; 51(6):706-712; Cour M., Klouche K., Souweine B., Quenot J. P., Schwebel C., Perinel S., et al. “Remote ischemic conditioning in septic shock: The RECO-sepsis randomized clinical trial”. Intensive Care Med. 2022 November; 48(11):1563-1572).

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment.

As used herein, the term “reduce” or other forms of the word, such as “reducing” or “reduction,” is meant lowering of an event or characteristic (e.g., vascular permeability). It is understood that this is typically in relation to some standard or expected value, in other words it is relative, but that it is not always necessary for the standard or relative value to be referred to. As used herein, the terms “inhibit” and “inhibition” also refer to a reduction or prevention of vascular leakage or inappropriate vascular permeability. Vascular permeability is considered to be “reduced” when vascular permeability is reduced by at least 10% in a given permeability assay and preferably at least 20%, 30%, 40%, 50%, 60%, 70%, 80%, 90% or more, up to and including complete inhibition, or 100%. Methods for assessing vascular permeability are well known in the art (see e.g. Wollborn, Jakob, et al. “Diagnosing capillary leak in critically ill patients: development of an innovative scoring instrument for non-invasive detection.” Annals of Intensive Care 11.1 (2021): 1-13.).

As used herein, the term “prevent” or other forms of the word, such as “preventing” or “prevention,” is meant to stop a particular event or characteristic (e.g., vascular permeability), to stabilize or delay the development or progression of a particular event or characteristic (e.g., vascular permeability), or to minimize the chances that a particular event or characteristic (e.g., vascular permeability) will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

As used herein, the term “therapeutically efficient amount” is intended an amount of pharmaceutical composition of the present invention administered to a patient that is sufficient to constitute a treatment as defined above.

### Methods

Accordingly, the first object of the present invention relates to a method of treating vascular permeability in a patient in need thereof comprising administering to the patient a therapeutically effective amount of i) an AREG polypeptide or ii) a polynucleotide encoding for an AREG polypeptide.

The method of the present invention is particularly suitable for reducing and/or preventing vascular permeability in a patient in need thereof. More particularly, the method of the present invention is suitable for reducing and/or preventing excessive vascular permeability and hyperpermeability. Even more particularly, the method of the present invention is suitable for the reducing and/or preventing capillary hyperpermeability.

In some embodiments, the patient suffers from a vascular permeability-associated disease or condition. Vascular permeability-associated diseases and conditions to which embodiments of the invention relate include, but are not necessarily limited to, oedema, cardiovascular disease, myocardial infarction, peripheral vascular disease, ischaemia, stroke, cancer, atherosclerosis, psoriasis, diabetes, autoimmune diseases such as rheumatoid arthritis, thrombocytopenia, altitude sickness, barotrauma, iatrogenic disorders, bacterial infections, viral infections, and ocular conditions associated with vascular leak such as nonproliferative and proliferative retinopathies (including diabetic retinopathy), macular oedema (including diabetic macular oedema), glaucoma and macular degeneration (including age-related macular degeneration). The oedema may be generalised oedema or localized or organ-specific oedema. The oedema may be, for example, cardiac oedema, pulmonary oedema, renal oedema, macular oedema, cerebral oedema, malnutritional oedema or lymphoedema. The oedema may result from a surgical procedure, in particular a major surgical procedure, such as cardiac surgery, organ transplantation surgery, knee and hip replacement surgery, dental surgery or limb amputation surgery (for example associated with diabetic complications).

In some embodiments, the patient suffers from a vascular leakage syndrome.

In some embodiments, the patient suffers from a systemic capillary leak syndrome.

In some embodiments, the vascular permeability is secondary to a sepsis.

As used herein, the term “sepsis” has its general meaning in the art and is a syndrome of physiologic, pathologic, and biochemical abnormalities induced by infection (Singer, Mervyn, et al. “The third international consensus definitions for sepsis and septic shock (Sepsis-3).” Jama 315.8 (2016): 801-810).

In some embodiments, the patient suffers from a SIRS. As used herein the term “SIRS” has its general meaning in the art and refers to systemic inflammatory response syndrome.

In some embodiments, the septic patient suffers from acute respiratory distress syndrome. As used herein, the term “acute respiratory distress syndrome” (abbreviated ARDS) relates to a severe, life-threatening medical condition characterized by presence of a risk factor (e.g. pneumoniapancreatitis, etc.), bilateral pulmonary infiltrates, and oxygen impairment not fully explained by cardiac failure. More specifically, the term ARDS as used herein relates to acute respiratory distress syndrome as convened in 2011 in the Berlin definition (ARDS Definition Task Force et al. 2012 JAMA 307(23): 2526-2533).

In some embodiments, the patient suffers from a shock. As used herein, the term “shock” is used herein, unless otherwise indicated, it is used to describe circulatory shock, cardiogenic shock, ischemic shock, hypervolemic shock, hemorrhagic shock, septic shock or other types of shock (for example post-resuscitation syndrome) associated with a reduction of blood volume in an organ or tissue, or an insufficient supply of blood to an organ or tissue. Shock that can be treated in accordance with the present invention can occur in a number of situations. For example, an event that creates a risk of shock can occur in civilian and military trauma settings, such as hemorrhage creating a risk of hemorrhagic shock. For another example, an event such as a planned surgery can create a risk of shock. Examples of such surgeries include, heart valve replacement surgeries, coronary artery bypass graft surgeries, stint placement surgeries, orthopedic surgeries, organ repair surgeries, organ transplantation surgeries, surgeries to implant devices, and the like.

In particular, the method of the present invention is particularly suitable for improving chances for return of spontaneous circulation (ROSC) after a cardiac arrest.

In some embodiments, the method of the present invention is particularly suitable for treating cardiac arrest-induced vascular permeability.

In some embodiments, the method of the present invention is particularly suitable for reducing and/or preventing vascular permeability during the treatment of ischemic conditions.

In some embodiments, the method of the present invention is particularly suitable for reducing and/or preventing vascular permeability that could occur after acute myocardial infarction.

In some embodiments, the method of the present invention is performed sequentially or concomitantly with a standard method for treating ischemic conditions. Typically, standard methods include reperfusion of the ischemic organ (e.g. heart) by angioplasty (e.g.; coronary, renal or carotid angioplasty), thrombolysis or coronary surgery. The term “percutaneous coronary intervention” means coronary angioplasty which is a therapeutic procedure to treat the stenotic (narrowed) coronary arteries of the heart found in coronary heart disease. The term “thrombolysis” means the administration of thrombolytic agents. Currently available thrombolyic agents include reteplase (r-PA or Retavase), alteplase (t-PA or Activase), urokinase (Abbokinase), prourokinase, anisoylated purified streptokinase activator complex (APSAC), and streptokinase.

In some aspects, the present invention relates to a method of treating an ischemic condition in a patient in need thereof comprising the steps consisting of i) restoring blood supply in the ischemic tissue, and reducing and/or preventing vascular permeability by administering to said patient a therapeutically effective amount of an AREG polypeptide or ii) a polynucleotide encoding for an AREG polypeptide, where steps i) and ii) are performed sequentially or concomitantly.

### AREG Polypeptides:

In some embodiments, the AREG polypeptide of the present invention comprises an amino acid sequence having at least 80% of identify with the amino acid sequence that ranges from the amino acid residue at position 142 to the amino acid residue at position 182 in SEQ ID NO:1 (“EGF domain”).

In some embodiments, the AREG polypeptide of the present invention comprises the amino acid sequence that ranges from the amino acid residue at position 142 to the amino acid residue at position 182 in SEQ ID NO:1 (“EGF domain”) and may differ from said amino acid sequence by 1, 2, 3, 4, 5, 6, 7, 8, 9, 10, 11, 12, 13, 14 or 15 substitutions, deletions and/or insertions.

According to the present invention the AREG polypeptide of the present invention is a soluble polypeptide. As used herein, the term “soluble polypeptide” means a polypeptide that is not membrane bound.

In some embodiments, the AREG polypeptide of the present invention is an 11.3 kDa glycoprotein that consists of 98 amino acid residues. In some embodiments, the AREG polypeptide of the present invention comprises or consists of an amino acid sequence having at least 80% of identity with the amino acid that ranges from the amino acid residue (S) at position 101 to amino acid residue (K) at position 198 in SEQ ID NO:1.

In some embodiments, the AREG polypeptide of the present invention is fully or partially glycosylated. As used herein, the term “glycosylated” with respect to a polypeptide means that a carbohydrate moiety is present at one or more sites of the protein molecule. In particular, a glycosylated protein refers to a protein that is typically modified by N-glycan or O-glycan addition. The term “fully glycosylated” indicates that all predetermined sites (i.e. the amino acid residues) in the polypeptide are glycosylated. The term “partially glycosylated” that one or more sites but not all are glycosylated.

Commercially sources of AREG polypeptides are well known and typically include those available from R&D Systems (Catalog number: 262-AR-100) or from Preprotech (Catalog Number: 100-55B).

In some embodiments, it is contemplated that the AREG polypeptides of the invention are modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution. A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain. Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications.

In some embodiments, the AREG polypeptide of the invention is fused a Fc domain of an immunoglobulin. Suitable immunoglobins are IgG, IgM, IgA, IgD, and IgE. IgG and IgA are preferred IgGs are most preferred, e.g. an IgG1. Said Fc domain may be a complete Fc domain or a function-conservative variant thereof. The AREG polypeptide of the invention may be linked to the Fc domain by a linker. The linker may consist of about 1 to 100, preferably 1 to 10 amino acid residues.

According to the invention, the AREG polypeptide of the invention may be produced by conventional automated peptide synthesis methods or by recombinant expression. General principles for designing and making proteins are well known to those of skill in the art. The AREG polypeptides of the invention may be synthesized in solution or on a solid support in accordance with conventional techniques. Various automatic synthesizers are commercially available and can be used in accordance with known protocols as described in Stewart and Young; Tam et al., 1983; Merrifield, 1986 and Barany and Merrifield, Gross and Meienhofer, 1979. The AREG polypeptides of the invention may also be synthesized by solid-phase technology employing an exemplary peptide synthesizer such as a Model 433A from Applied Biosystems Inc. The purity of any given protein; generated through automated peptide synthesis or through recombinant methods may be determined using reverse phase HPLC analysis. Chemical authenticity of each peptide may be established by any method well known to those of skill in the art. As an alternative to automated peptide synthesis, recombinant DNA technology may be employed wherein a nucleotide sequence which encodes a protein of choice is inserted into an expression vector, transformed or transfected into an appropriate host cell and cultivated under conditions suitable for expression as described herein below. Recombinant methods are especially preferred for producing longer polypeptides. A variety of expression vector/host systems may be utilized to contain and express the peptide or protein coding sequence. These include but are not limited to microorganisms such as bacteria transformed with recombinant bacteriophage, plasmid or cosmid DNA expression vectors; yeast transformed with yeast expression vectors (Giga-Hama et al., 1999); insect cell systems infected with virus expression vectors (e.g., baculovirus, see Ghosh et al., 2002); plant cell systems transfected with virus expression vectors (e.g., cauliflower mosaic virus, CaMV; tobacco mosaic virus, TMV) or transformed with bacterial expression vectors (e.g., Ti or pBR322 plasmid; see e.g., Babe et al., 2000); or animal cell systems. Those of skill in the art are aware of various techniques for optimizing mammalian expression of proteins, see e.g., Kaufman, 2000; Colosimo et al., 2000. Mammalian cells that are useful in recombinant protein productions include but are not limited to VERO cells, HeLa cells, Chinese hamster ovary (CHO) cell lines, COS cells (such as COS-7), W138, BHK, HepG2, 3T3, RIN, MDCK, A549, PC12, K562 and 293 cells. Exemplary protocols for the recombinant expression of the peptide substrates or fusion polypeptides in bacteria, yeast and other invertebrates are known to those of skill in the art and a briefly described herein below. Mammalian host systems for the expression of recombinant proteins also are well known to those of skill in the art. Host cell strains may be chosen for a particular ability to process the expressed protein or produce certain post-translation modifications that will be useful in providing protein activity. Such modifications of the polypeptide include, but are not limited to, acetylation, carboxylation, glycosylation, phosphorylation, lipidation and acylation. Post-translational processing which cleaves a “prepro” form of the protein may also be important for correct insertion, folding and/or function. Different host cells such as CHO, HeLa, MDCK, 293, WI38, and the like have specific cellular machinery and characteristic mechanisms for such post-translational activities and may be chosen to ensure the correct modification and processing of the introduced, foreign protein. In the recombinant production of the AREG polypeptides of the invention, it would be necessary to employ vectors comprising polynucleotide molecules for encoding the AREG polypeptides of the invention. Methods of preparing such vectors as well as producing host cells transformed with such vectors are well known to those skilled in the art. The polynucleotide molecules used in such an endeavor may be joined to a vector, which generally includes a selectable marker and an origin of replication, for propagation in a host. These elements of the expression constructs are well known to those of skill in the art. Generally, the expression vectors include DNA encoding the given protein being operably linked to suitable transcriptional or translational regulatory sequences, such as those derived from a mammalian, microbial, viral, or insect genes. Examples of regulatory sequences include transcriptional promoters, operators, or enhancers, mRNA ribosomal binding sites, and appropriate sequences which control transcription and translation.

### Polynucleotides:

In some embodiments, the polynucleotide of the present invention is a messenger RNA (mRNA).

In some embodiments, the polynucleotide is inserted in a vector, such a viral vector.

As used herein, the terms “vector” refers to the vehicle by which a polynucleotide can be introduced into a host cell, so as to transform the host and promote expression (e.g., transcription and translation) of the introduced sequence. As used herein, the term “viral vector” encompasses vector DNA as well as viral particles generated thereof. Viral vectors can be replication-competent, or can be genetically disabled so as to be replication-defective or replication-impaired. The term “replication-competent” as used herein encompasses replication-selective and conditionally-replicative viral vectors which are engineered to replicate better or selectively in specific host cells (e.g. tumoral cells). As used herein, the term “non-viral vector” notably refers to a vector of plasmid origin, and optionally such a vector combined with one or more substances improving the transfectional efficiency and/or the stability of said vector and/or the protection of said vector.

In some embodiments, the viral vector is a AAV vector. As used herein, the term “AAV vector” means a vector derived from an adeno-associated virus serotype, including without limitation, AAV1, AAV2, AAV3, AAV4, AAV5, AAV6, AAV7, AAV8, AAV9, and mutated forms thereof. AAV vectors can have one or more of the AAV wild-type genes deleted in whole or part, preferably the rep and/or cap genes, but retain functional flanking ITR sequences.

In some embodiments, the viral vector is a retroviral vector. As used herein, the term “retroviral vector” refers to a vector containing structural and functional genetic elements that are primarily derived from a retrovirus. In some embodiments, the retroviral vector of the present invention derives from a retrovirus selected from the group consisting of alpharetroviruses (e.g., avian leukosis virus), betaretroviruses (e.g., mouse mammary tumor virus), gammaretroviruses (e.g., murine leukemia virus), deltaretroviruses (e.g., bovine leukemia virus), epsilonretroviruses (e.g., Walley dermal sarcoma virus), lentiviruses (e.g., HIV-1, HIV-2) and spumaviruses (e.g., human spumavirus).

In some embodiments, the retroviral vector of the present invention is a replication deficient retroviral virus particle, which can transfer a foreign imported RNA of a gene instead of the retroviral mRNA.

In some embodiments, the retroviral vector of the present invention is a lentiviral vector.

As used herein, the term “lentiviral vector” refers to a vector containing structural and functional genetic elements that are primarily derived from a lentivirus. In some embodiments, the lentiviral vector of the present invention is selected from the group consisting of HIV-1, HIV-2, SIV, FIV, EIAV, BIV, VISNA and CAEV vectors. In some embodiments, the lentiviral vector is a HIV-1 vector.

The structure and composition of the vector genome used to prepare the retroviral vectors of the present invention are in accordance with those described in the art. Especially, minimum retroviral gene delivery vectors can be prepared from a vector genome, which only contains, apart from the recombinant nucleic acid molecule of the present invention, the sequences of the retroviral genome which are non-coding regions of said genome, necessary to provide recognition signals for DNA or RNA synthesis and processing. In some embodiment, the retroviral vector genome comprises all the elements necessary for the nucleic import and the correct expression of the polynucleotide of interest (i.e. the transgene). As examples of elements that can be inserted in the retroviral genome of the retroviral vector of the present invention are at least one (preferably two) long terminal repeats (LTR), such as a LTR5′ and a LTR3′, a psi sequence involved in the retroviral genome encapsidation, and optionally at least one DNA flap comprising a cPPT and a CTS domains. In some embodiments of the present invention, the LTR, preferably the LTR3′, is deleted for the promoter and the enhancer of U3 and is replaced by a minimal promoter allowing transcription during vector production while an internal promoter is added to allow expression of the transgene. In particular, the vector is a Self-INactivating (SIN) vector that contains a non-functional or modified 3′ Long Terminal Repeat (LTR) sequence. This sequence is copied to the 5′ end of the vector genome during integration, resulting in the inactivation of promoter activity by both LTRs. Hence, a vector genome may be a replacement vector in which all the viral coding sequences between the 2 long terminal repeats (LTRs) have been replaced by the recombinant nucleic acid molecule of the present invention.

In some embodiments, the retroviral vector genome is devoid of functional gag, pol and/or env retroviral genes. By “functional” it is meant a gene that is correctly transcribed, and/or correctly expressed. Thus, the retroviral vector genome of the present invention in this embodiment contains at least one of the gag, pol and env genes that is either not transcribed or incompletely transcribed; the expression “incompletely transcribed” refers to the alteration in the transcripts gag, gag-pro or gag-pro-pol, one of these or several of these being not transcribed. In some embodiments, the retroviral genome is devoid of gag, pol and/or env retroviral genes.

In some embodiments the retroviral vector genome is also devoid of the coding sequences for Vif-, Vpr-, Vpu- and Nef-accessory genes (for HIV-1 retroviral vectors), or of their complete or functional genes.

Typically, the retroviral vector of the present invention is non replicative i.e., the vector and retroviral vector genome are not able to form new particles budding from the infected host cell. This may be achieved by the absence in the retroviral genome of the gag, pol or env genes, as indicated in the above paragraph; this can also be achieved by deleting other viral coding sequence(s) and/or cis-acting genetic elements needed for particles formation.

Thus the present invention encompasses use of virus-like particles. As used herein, the term “virus-like particle” or “VLP” refers to a structure resembling a virus particle but devoid of the viral genome, incapable of replication and devoid of pathogenicity. The particle typically comprises at least one type of structural protein from a virus. Preferably only one type of structural protein is present. Most preferably no other non-structural component of a virus is present. Thus, virus-like particles can be spontaneously self-assembled by viral structural proteins under appropriate conditions in vitro while excluding the genetic material and potential replication probability. virus-like particles, with a diameter of approximately 20 to 150 nm, also have the characteristics of nanometer materials, such as large surface area, surface-accessible amino acids with reactive moieties (e.g., lysine and glutamic acid residues), inerratic spatial structure, and good biocompatibility. Therefore, assembled virus-like particles have great potential as a delivery system for specifically carrying a variety of cargos. In some embodiments, one or more of the zinc finger motifs of the Gag protein is/are substituted by one or more RNA-binding domain(s). In some embodiments, the RNA-binding domain is the Coat protein of the MS2 bacteriophage, of the PP7 phage or of the Q3 phage, the prophage HK022 Nun protein, the U1A protein or the hPum protein. More preferably, the RNA binding domain is the Coat protein of the MS2 bacteriophage or of the PP7 phage. Even more preferably the RNA-binding domain is the Coat protein of the MS2 bacteriophage. These embodiments are particularly suitable for packaging the mRNA encoding for the apelin polypeptide into the VLP. Thus, in some embodiments, the mRNA encoding for the apelin polypeptide that is encapsuled in the virus particle of the present invention comprises at least one encapsidation sequence. By “encapsidation sequence” is meant an RNA motif (sequence and three-dimensional structure) recognized specifically by an RNA-binding domain as above described. Preferably, the encapsidation sequence is a stem-loop motif. Even more preferably, the encapsidation sequence of the retroviral particle is the stem-loop motif of the RNA of the MS2 bacteriophage or of the PP7 phage such as. The stem-loop motif and more particularly the stem-loop motif of the RNA of the MS2 bacteriophage or that of the RNA of the PP7 phage may be used alone or repeated several times, preferably from 2 to 25 times, more preferably from 2 to 18 times, for example from 6 to 18 times. In some embodiments, the present invention encompasses the use of the LentiFlash® technology that based on non-integrative lentiviral particles constructed using a bacteriophage coat protein and its cognate 19-nt stem loop, to replace the natural lentiviral Psi packaging sequence, in order to achieve active mRNA packaging into the lentiviral particles (Prel A, Caval V, Gayon R, Ravassard P, Duthoit C, Payen E, Maouche-Chretien L, Creneguy A, Nguyen T H, Martin N, Piver E, Sevrain R, Lamouroux L, Leboulch P, Deschaseaux F, Bouilld P, Sensbd L, Pagds J C. Highly efficient in vitro and in vivo delivery of functional RNAs using new versatile MS2-chimeric retrovirus-like particles. Mol Ther Methods Clin Dev. 2015 Oct. 21; 2:15039. doi: 10.1038 mtm.2015.39. PMID: 26528487; PMCID: PMC4613645).

The retroviral vectors of the present invention can be produced by any well-known method in the art including by transfection (s) transient (s), in stable cell lines and/or by means of helper virus.

### Formulations:

In some embodiments, the polypeptide or polynucleotide of the present invention can be conjugated to at least one other molecule. Typically, said molecule is selected from the group consisting of polynucleotides, polypeptides, lipids, lectins, carbohydrates, vitamins, cofactors, and drugs. In some embodiments, the polypeptide or polynucleotide of the present invention is formulated using one or more lipid-based structures that include but are not limited to liposomes, lipoplexes, or lipid nanoparticles (Paunovska, Kalina, David Loughrey, and James E. Dahlman. “Drug delivery systems for RNA therapeutics.” Nature Reviews Genetics (2022): 1-16). Liposomes are artificially-prepared vesicles which can primarily be composed of a lipid bilayer and can be used as a delivery vehicle for the administration of pharmaceutical formulations. Liposomes can be of different sizes such as, but not limited to, a multilamellar vesicle (MLV) which can be hundreds of nanometers in diameter and can contain a series of concentric bilayers separated by narrow aqueous compartments, a small unicellular vesicle (SUV) which can be smaller than 50 nm in diameter, and a large unilamellar vesicle (LUV) which can be between 50 and 500 nm in diameter. Liposome design can include, but is not limited to, opsonins or ligands in order to improve the attachment of liposomes to unhealthy tissue or to activate events such as, but not limited to, endocytosis. Liposomes can contain a low or a high pH in order to improve the delivery of the pharmaceutical formulations. As a non-limiting example, liposomes such as synthetic membrane vesicles are prepared by the methods, apparatus and devices described in US Patent Publication No. US20130177638, US20130177637, US20130177636, US20130177635, US20130177634, US20130177633, US20130183375, US20130183373 and US20130183372. In some embodiments, the liposomes are formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (as described in US20100324120) and liposomes which can deliver small molecule drugs such as, but not limited to, DOXIL® from Janssen Biotech, Inc. (Horsham, Pa.). The polypeptide of polynucleotide of the present invention can be encapsulated by the liposome and/or it can be contained in an aqueous core which can then be encapsulated by the liposome (see International Pub. Nos. WO2012031046, WO2012031043, WO2012030901 and WO2012006378 and US Patent Publication No. US20130189351, US20130195969 and US20130202684). In some embodiments, the polynucleotide of the present invention is formulated with stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999 6:271-281; Zhang et al. Gene Therapy. 1999 6:1438-1447; Jeffs et al. Pharm Res. 2005 22:362-372; Morrissey et al., Nat Biotechnol. 2005 2:1002-1007; Zimmermann et al., Nature. 2006 441:111-114; Heyes et al. J Contr Rel. 2005 107:276-287; Semple et al. Nature Biotech. 2010 28:172-176; Judge et al. J Clin Invest. 2009 119:661-673; deFougerolles Hum Gene Ther. 2008 19:125-132; U.S. Patent Publication No US20130122104).

Typically the active ingredient of the present invention (i.e. the polypeptide or polynucleotide) is combined with pharmaceutically acceptable excipients, and optionally sustained-release matrices, such as biodegradable polymers, to form pharmaceutical compositions. The term “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The invention will be further illustrated by the following figures and examples. However, these examples and figures should not be interpreted in any way as limiting the scope of the present invention.

### FIGURES

FIG. 1. Link between AREG concentration in the plasma at ICU arrival, and the fluid balance during 72 hours, in an independent cohort of 77 cardiogenic and post-cardiac arrest patients. ***p<0.001.

FIG. 2. (A) Total weight/dry weight ratio of various organs after one hour post-ROSC, in the murine model of cardiac arrest. (B) Serum Amphiregulin concentration at one hour after ROSC. *p<0.05.

FIG. 3. Survival of areg-KO, wild-type, and wild-type mice injected with recombinant mouse AREG at the time of resuscitation (n>10 per group).

FIG. 4. Time-course of left ventricular ejection fraction (LVEF) after cardiac arrest, in wild-type mice injected with recombinant mouse AREG at the time of resuscitation, and their littermate controls. T0=ROSC.

FIG. 5. Intra-organs fluorescence ratio of fluorescent-labeled dextran (155 kDa) and Cadaverine in the organs of wild-type mice injected with recombinant mouse AREG at the time of resuscitation, and their littermate controls. Organs were harvested after 4 minutes of Low-Flow (during resuscitation), and 4 minutes of circulating i.v.-injected dyes. Ratios of heart, lungs, right kidney, liver and brain were normalized on the mean control value for each organ, and pooled as a total score ratio. n=4 per group. *p<0.05 between groups. FI, Fluorescence Intensity.

### EXAMPLE

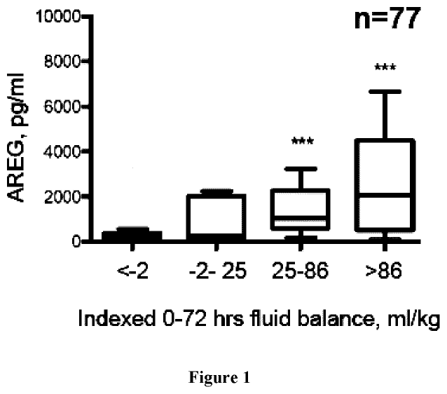

In a prospective translational study, we performed RNAseq in CD14+ circulating monocytes from 11 patients (with similar confounding factors) exhibiting very severe cardiogenic shock (mean age 55, IQR 33-66). All were under venous-arterial extracorporeal membrane oxygenation (ECMO), with SAPSII score of 84 (53-107), pH 7.0 (6.9-7.2) and lactatemia at 11 (9-13) mmol/L at ECMO implantation. Comparison of RNAseq in patients who developed severe capillary leakage (arbitrarily defined as a fluid balance>75 ml/kg in the first 72 h, n=7) and patients who did not (n=4) revealed 860 differentially expressed genes. 38 genes were retained after false discovery rate correction. Of particular interest was Amphiregulin (Areg), 56-fold more highly expressed in monocytes of patients with massive vascular leakage. Levels of circulating AREG at ICU inclusion was confirmed correlated with the level of fluid balance in an independent cohort of 77 patients with circulatory failure (cardiogenic and post-resuscitation syndrome) (FIG. 1).

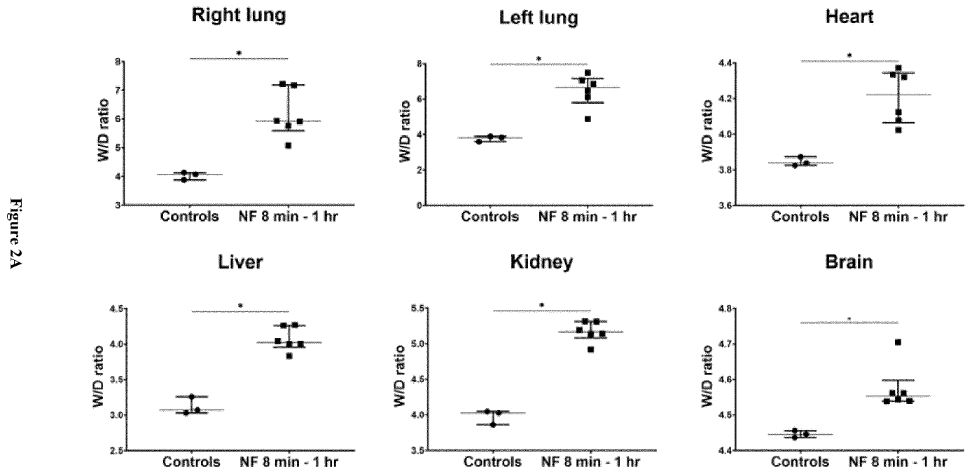

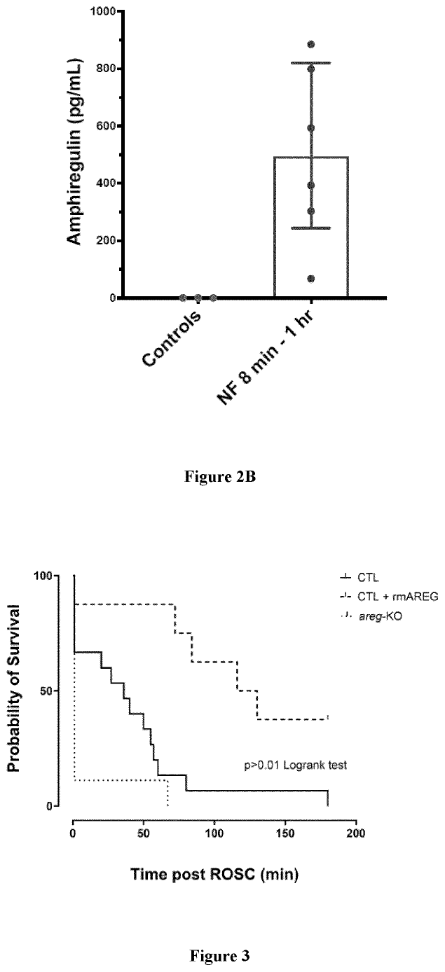

We have developed a mouse model of resuscitated cardiac arrest (CA). Briefly, after insertion of a catheter into the jugular vein, CA is induced by KCl injection. Mice are intubated, and return of spontaneous circulation is acquired by resuscitation manoeuvres and epinephrine injection after a period of no-flow. We have confirmed a marked oedema in organs and a large increase in expression of circulating AREG (FIG. 2). In a pig model of post-CA dysfunction with extracorporeal resuscitation that requires a dramatic increase in fluid requirements to maintain blood pressure (which corresponds to the situation in which our RNAseq demonstrated Areg expression in humans), we also report massively induced expression of circulating AREG.

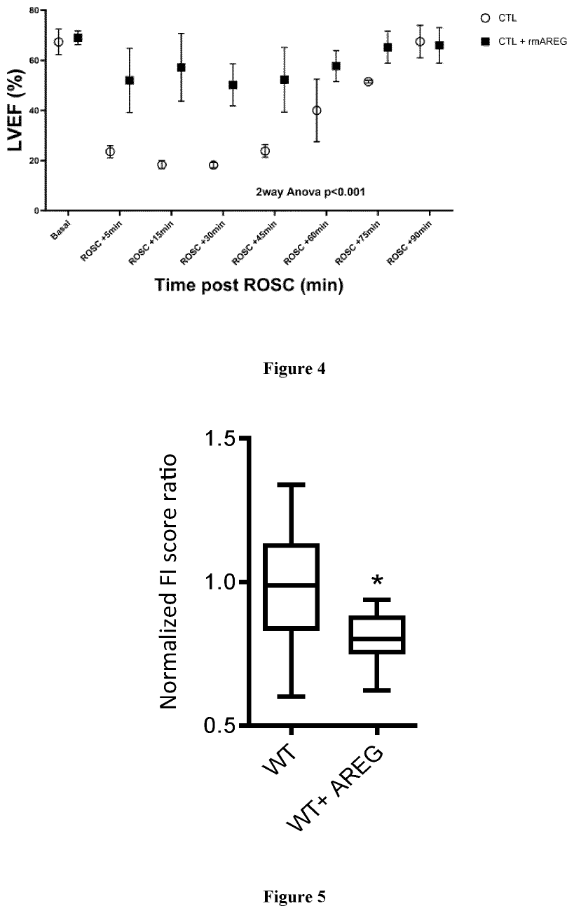

Most importantly, we showed a crucial role for this protein in Areg−/− mice, with i) less KO-mice achieving a return of spontaneous circulation (ROSC) vs. in WT mice (10% vs. 80%, n>10 per group, p<0.05) and ii) a beneficial effect of 10 μg recombinant AREG i.v. injection at the time of resuscitation, with a trend toward higher rate of ROSC, and a significantly better survival, compared to WT mice (FIG. 3). Echocardiographic studies demonstrated a better myocardial function after cardiac arrest, in mice injected with 10 μg recombinant AREG at the time of resuscitation (FIG. 4). Extravasation of fluorescent labeled dextran (155 kDa) and Cadaverine in the organs of wild-type mice injected with recombinant mouse AREG at the time of resuscitation, and their littermate controls, confirmed a reduced vascular leakage of large molecules in recombinant AREG-injected mice (FIG. 5). The results thus indicate that administering AREG in a patient suffering from vascular permeability would be beneficial, all the more than the pharmacokinetics profile of the protein seems to be very promising (i.e. the half-life of the protein after 10 μg i.p. injection reaches 110 min).

### REFERENCES

Throughout this application, various references describe the state of the art to which this invention pertains. The disclosures of these references are hereby incorporated by reference into the present disclosure.

1. A method of treating vascular hyperpermeability in a patient in need thereof comprising administering to the patient a therapeutically effective amount of i) an AREG polypeptide or ii) a polynucleotide encoding the AREG polypeptide.

2. The method of claim 1 wherein the patient suffers from a vascular hyperpermeability-associated disease or condition selected from the group consisting of limited oedema, cardiovascular disease, myocardial infarction, peripheral vascular disease, ischaemia, stroke, cancer, atherosclerosis, psoriasis, diabetes, an autoimmune disease thrombocytopenia, altitude sickness, barotrauma, an iatrogenic disorder, a bacterial infection, a viral infection, and an ocular condition associated with vascular leakage.

3. The method of claim 1 wherein the patient suffers from a vascular leakage syndrome.

4. The method of claim 1 wherein the patient suffers from a systemic capillary leak syndrome.

5. The method of claim 1 wherein the vascular hyperpermeability is secondary to a sepsis.

6. The method of claim 1 wherein the patient suffers from a SIRS.

7. The method of claim 1 wherein the patient suffers from a shock.

8. The method of claim 8 wherein the shock is selected from the group consisting of circulatory shock, cardiogenic shock, ischemic shock, hypervolemic shock, hemorrhagic shock, and septic shock.

9. The method of claim 1 wherein the method improves a return of spontaneous circulation (ROSC) after a cardiac arrest.

10. The method of claim 1 wherein the method treats cardiac arrest-induced vascular hyperpermeability.

11. The method of claim 1 wherein the method preserves vascular endothelial cell barrier integrity during the treatment of ischemic conditions.

12. A method of treating an ischemic condition in a patient in need thereof comprising the steps consisting of i) restoring blood supply in ischemic tissue, and ii) preserving vascular endothelial cell barrier integrity of said ischemic tissue by administering to said patient a therapeutically effective amount of an AREG polypeptide or a polynucleotide encoding the AREG polypeptide, where steps i) and ii) are performed sequentially or concomitantly.

13. The method according to claim 1, wherein the AREG polypeptide comprises an amino acid sequence having at least 80% identify with the amino acid sequence that ranges from the amino acid residue at position 142 to the amino acid residue at position 182 in SEQ ID NO:1.

14. The method of claim 13 wherein the AREG polypeptide comprises the amino acid sequence that ranges from the amino acid residue at position 142 to the amino acid residue at position 182 in SEQ ID NO:1 and optionally differs from said amino acid sequence by 1, 2, 3, 4, 5, 6, 7, 8, 9, 10, 11, 12, 13, 14 or 15 substitutions, deletions and/or insertions.

15. The method of claim 14 wherein the AREG polypeptide comprises or consists of an amino acid sequence having at least 80% identity with the amino acid that ranges from the amino acid residue (S) at position 101 to amino acid residue (K) at position 198 in SEQ ID NO:1.

16. The method of claim 14 wherein the AREG polypeptide is fully or partially glycosylated.

17. The method according to claim 1, wherein the polynucleotide is a messenger RNA (mRNA).

18. The method of claim 2, wherein the autoimmune disease is rheumatoid arthritis.

19. The method of claim 2, wherein the ocular condition associated with vascular leakage is a nonproliferative or proliferative retinopathy, macular oedema, glaucoma or macular degeneration.

20. The method of claim 19, wherein the nonproliferative or proliferative retinopathy is diabetic retinopathy, the macular oedema is diabetic macular oedema and the macular degeneration is age-related macular degeneration.


SEQUENCE LISTING (1 sequence)

SEQ ID NO: 1 - protein, 252 aa, Homo sapiens
MRAPLLPPAPVVLSLLILGSGHYAAGLDLNDTYSGKREPFSGDHSADGFEVTSRSEMSSGSEISPVSEMPSSSEPSSGADYDYSEEYDNEPQIPGYIVDDSVRVEQVVKPPQNKTESENTSDKPKRKKKGGKNGKNRRNRKKKNPCNAEFQNFCIHGECKYIEHLEAVTCKCQQEYFGERCGEKSMKTHSMIDSSLSKIALAAIAAFMSAVILTAVAVITVQLRRQYVRKYEGEAEERKKLRQENGNVHAIA